SEQUENCE LISTING (4 sequences)

SEQ ID NO: 1 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 2 - protein, 100 aa, Cannabis sativa
MAVKHLIVLKFKDEITEAQKEEFFKTYVNLVNIIPAMKDVYWGKDVTQKKEEGYTHIVEVTFESVETIQDYIIHPAHVGFGDVYRSFWEKLLIFDYTPRK

SEQ ID NO: 3 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 4 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

(19) United States
(12) Patent Application Publication
(10) Pub. No.: US 2023/0279452 A1
(43) Pub. Date: Sep. 7, 2023

(54) PREPARING AND MODIFYING MEROTERPENE POLYKETIDES, KETONES, AND LACTONES FOR CANNABINOID SEMISYNTHESIS

(71) Applicant: LYGOS, INC., Berkeley, CA (US)

(72) Inventors: NICHOLAS OHLER, Berkeley, CA (US); JASON POULOS, Berkeley, CA (US); CHI LE, Berkeley, CA (US); NEIL MCALPINE, Berkeley, CA (US); ANTHONY FARINA, Berkeley, CA (US)

(21) Appl. No.: 17/799,414

(22) PCT Filed: Feb. 9, 2021
(86) PCT No.: PCT/US2021/017226

§ 371 Date: Aug. 12, 2022

Related U.S. Application Data
(60) Provisional application No. 62/975,378, filed on Feb. 12, 2020.
(60) Provisional application No. 63/019,098, filed on May 1, 2020.
(60) Provisional application No. 63/122,360, filed on Dec. 7, 2020.

(51) Int. Cl. C12P 17/06; C12N 15/52; C12P 7/22

(52) U.S. Cl. CPC C12P 17/06; C12N 15/52; C12P 7/22; C12Y 203/01206; C12Y 404/01026; C12Y 602/01002

(57) ABSTRACT

Provided herein are processes, including semi-synthetic, and synthetic processes for preparing cannabinoids, and cannabinoid compositions provided thereby.

### STATEMENT ABOUT FEDERAL FUNDING

Not applicable.

### PRIORITY CLAIM

This application is a U.S. National Phase Application of International Application No. PCT/US2021/017226 filed Feb. 9, 2021, which claims priority to and the benefit of U.S. Provisional Application Nos. U.S. 62/975,378 filed Feb. 12, 2020; U.S. 63/019,098 filed May 1, 2020; and U.S. 63/122,360 filed Dec. 7, 2020, each of which is incorporated herein in its entirety by reference.

### SEQUENCE LISTING

The instant application contains a Sequence Listing which has been submitted electronically in ASCII format and is hereby incorporated by reference in its entirety. Said ASCII copy, created on Feb. 8, 2021, is named LYGOS-0040-01-WO_SL.txt and is 11,773 bytes in size.

### REFERENCE TO SEQUENCE LISTING

The present application is filed with sequence listings attached hereto and incorporated by reference, including Appendix A, titled “Sequence IDs”.

### FIELD

This invention relates at least in part to processes, including semi-synthetic, and synthetic processes for preparing meroterpene polyketides, ketones, and lactones, such as cannabinoids, and meroterpene compositions provided thereby.

### BACKGROUND

There is a need for processes, particularly semi-synthetic, and synthetic processes for preparing cannabinoids, and cannabinoid compositions provided thereby.

### SUMMARY

In certain aspects, provided herein are processes, including semi-synthetic, and synthetic processes for preparing cannabinoids, and cannabinoid compositions provided thereby. A semi-synthetic process refers to a process of preparing one or more cannabinoids, where a fermentation-based process is combined, preferably but not necessarily without separation, purification, solvent-swap, and the likes, with a chemical synthesis process.

In one aspect, provided herein is a process for preparing one or more of a compound of formula (IA), (IB), and (IC):

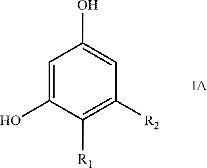

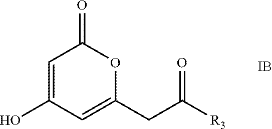

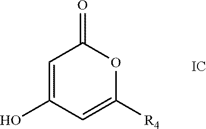

or a salt or an ester (carboxy and/or phenolic) thereof, wherein
R₁ is H or CO₂H;
each R₂, R₃, and R₄ is independently C₃-C₁₀ alkyl, C₃-C₁₀ alkenyl, or C₃-C₁₀ alkynyl, preferably, C₃-C₈ alkyl, more preferably, n-pentyl or n-propyl; the process comprising:
fermenting a recombinant microorganism comprising: a polyketide synthase, wherein the polyketide synthase combines an acyl-CoA and two or more, such as two or three, malonyl-CoA to produce a polyketide thereby preparing one or more of a compound of formula (IA), (IB), and (IC) or the salt or the ester thereof. Optionally a dimeric α+β barrel (DABB) protein is also co-expressed with the polyketide resulting in a polyketide comprising a carboxylic acid. In one embodiment, a compound of formula IA comprises aromatic polyketides (R₁=H). In one embodiment, a compound of formula IA comprises aromatic polyketides (R₁=CO₂H).

In one embodiment, the microorganism is fermented aerobically in the presence of a water immiscible, liquid, hydrophobic phase which dissolves the one or more of a compound of formula (IA), (IB), and (IC) or the salt or ester thereof. In another embodiment, the process further comprises separating the hydrophobic phase from an aqueous phase comprising the microorganism, the separating comprising a first continuous centrifugation to separate the cells and a bulk of a spent broth from the hydrophobic phase, followed by a second continuous centrifugation to separate the hydrophobic phase from the remaining aqueous phase. In another embodiment, the process further comprises: esterifying, isoprenylating, or performing an annulation of the compound included in the hydrophobic phase, under conditions suitable to perform an esterification, isoprenylation, or annulation without the need for a solvent swap.

In another aspect, provided herein is a process comprising:
aerobically fermenting a recombinant microorganism comprising: a polyketide synthase, optionally an olivetolic acid cyclase (OAC), and further optionally a hexanoyl Co-A synthetase (HCS), wherein the fermenting is performed in the presence of a water immiscible, liquid, hydrophobic phase,
to prepare one or more of: olivetolic acid or a salt or ester thereof, and olivetol or an ester thereof,
wherein the hydrophobic phase dissolves olivetolic acid or a salt or ester thereof or olivetol or an ester thereof.

In another aspect, provided herein is a process comprising:
aerobically fermenting a recombinant microorganism comprising: a polyketide synthase, optionally an olivetolic acid cyclase (OAC), and further optionally butyryl Co-A synthetase, wherein the fermenting is performed in the presence of a water immiscible, liquid, hydrophobic phase;
to prepare one or more of: divarinic acid or a salt or ester thereof, and divarin,
wherein the hydrophobic phase dissolves divarinic acid or a salt or ester thereof or divarin, as they are prepared.

In some embodiments, the microorganism comprises an olivetolic acid cyclase (OAC). In some embodiments, the microorganism comprises a hexanoyl Co-A synthetase (HCS). In other embodiments, a variety of acyl activating enzymes, which are well known to the skilled artisan, other than HCS or CsAAE1, are useful in accordance with this invention.

In one embodiment, 3 acyl -CoAs are combined.

In one embodiment, a compound of formula IA is provided. In one embodiment, a compound of formula IB is provided. In one embodiment, a compound of formula IC is provided.

In one embodiment, each R¹ independently is H. In one embodiment, each R¹ independently is CO₂H or a salt thereof.

In one embodiment, each R₂ is independently C₃-C₁₀ alkyl. In one embodiment, each R₂ is independently C₃-C₈ alkyl. In one embodiment, each R₂ is independently octyl. In one embodiment, each R₂ is independently pentyl. In one embodiment, each R₂ is independently C₃-C₁₀ propyl. In one embodiment, each R₂ is independently C₃-C₁₀ alkenyl. In one embodiment, each R₂ is independently C₃-C₁₀ alkynyl.

In one embodiment, each R₃ is independently C₃-C₁₀ alkyl. In one embodiment, each R₃ is independently C₃-C₈ alkyl. In one embodiment, each R₃ is independently octyl. In one embodiment, each R₃ is independently pentyl. In one embodiment, each R₃ is independently C₃-C₁₀ propyl. In one embodiment, each R₃ is independently C₃-C₁₀ alkenyl. In one embodiment, each R₃ is independently C₃-C₁₀ alkynyl.

In one embodiment, each R₄ is independently C₃-C₁₀ alkyl. In one embodiment, each R₄ is independently C₃-C₈ alkyl. In one embodiment, each R₄ is independently octyl. In one embodiment, each R₄ is independently pentyl. In one embodiment, each R₄ is independently C₃-C₁₀ propyl. In one embodiment, each R₄ is independently C₃-C₁₀ alkenyl. In one embodiment, each R₄ is independently C₃-C₁₀ alkynyl.

In some embodiments, the alkyl, alkenyl, or alkynyl groups are substituted with 1-3 substituents. Suitable substituents include halo, hydroxy, vinyl, ethynyl, and the likes.

### BRIEF DESCRIPTION OF FIGURES

FIG. 1 schematically illustrates recovery of olivetol and other compounds of formula IA in accordance with the present invention.

FIG. 2 schematically illustrates semisynthesis of cannabinoids (CBG) by prenylation of fermented olivetol.

FIG. 3 schematically illustrates semisynthesis of cannabinoids (CBC) by prenylation of fermented olivetol.

### DETAILED DESCRIPTION

While the present invention is described herein with reference to aspects and specific embodiments thereof, those skilled in the art will recognize that various changes may be made and equivalents may be substituted without departing from the invention. The present invention is not limited to particular nucleic acids, expression vectors, enzymes, host microorganisms, or processes, as such may vary. The terminology used herein is for purposes of describing particular aspects and embodiments only, and is not to be construed as limiting. In addition, many modifications may be made to adapt a particular situation, material, composition of matter, process, process step or steps, in accordance with the invention. All such modifications are within the scope of the claims appended hereto.

### Definitions

As used in the specification and the appended claims, the singular forms “a”, “an”, and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “ferment”, “fermentative”, and “fermentation” are used herein to describe culturing host cells and microbes under conditions to produce useful chemicals, including but not limited to conditions under which microbial growth, be it aerobic or anaerobic, occurs.

The terms “cell,” “host cell” “microorganism” and “host microorganism” are used interchangeably herein to refer to a living cell that can perform one or more steps of the cannabinoid pathway, e.g., as illustrated herein below. A host cell can be (or is) transformed via insertion of an expression vector. A host microorganism or cell as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g. greater than 50% or greater than 75%, or essentially, e.g., greater than 90%, 95%, 98% or 99%, free of components that normally accompany it in its native state, e.g., the state in which it is naturally found or the state in which it exists when it is first produced.

Polyketide synthases (PKSs) are a family of multi-domain enzymes or enzyme complexes that produce polyketides, a large class of secondary metabolites, in bacteria, fungi, plants, and a few animal lineages. The terms “polyketide synthase”, “PKS”, “olivetol synthase” (“OLS”), “tetraketide synthase”, TKS, and olivetolic synthase as described herein or elsewhere typically refers to any enzyme capable of converting three molecules of malonyl-CoA and one molecule of hexanoyl-CoA to olivetol. A wild type example of an OLS is the native C. sativa OLS enzyme (UniProt ID: B1Q2B6; SEQ ID NO: 1).

Sequence ID 1 :TKSMNHLRAEGPASVLAIGTANPENILlQDEFPDY
YFRVTKSEHMTQLKEKFRKICDKSMIRKRNCFLNEEHLKQNPRLVEHEMQ
TLDARQDMLVVEVPKLGKDACAKAIKEWGQPKSKITHLIFTSASTTDMPG
ADYHCAKLLGLSPSVKRVMMYQLGCYGGGTVLRIAKDIAENNKGARVLAV
CCDIMACLFRGPSDSDLELLVGQAIFGDGAAAVIVGAEPDESVGERPIFE
LVSTGQTILPNSEGTIGGHIREAGLIFDLHKDVPMLISNNIEKCLIEAFT
PIGISDWNSIFWITHPGGKAILDKVEEKLDLKKEKFVDSRHVLSEHGNMS
SSTVLFVMDELRKRSLEEGKSTTGDGFEWGVLFGFGPGLTVERVVVRSVP
IKY

The term “hexanoyl-CoA synthetase” (“HCS”) as used herein refers to any enzyme capable of catalyzing the conversion of hexanoate (a short-chain fatty acid anion that is the conjugate base of hexanoic acid, also known as caproic acid) or hexanoic acid, and a free CoA to hexanoyl-CoA. A non-limiting example of a hexanoyl-CoA synthetase is the FadK protein derived from E. coli.

The cannabinoid biosynthetic pathway utilizes a variety of enzymes, catalysts, and intermediate compounds. For example, cannabigerolic acid synthase (EC: 2.5.1.102) is used to convert OLA to cannabigerolic acid, which is a key intermediate acted upon by a variety of enzymes during THC synthesis. Cannabidiolic acid synthase (EC: 1.21.3.7) is used to convert cannabigerolic acid into cannabidiolic acid. Tetrahydrocannabinolic acid synthase (EC: 1.21.3.8) is used to convert cannabigerolic acid into Δ⁹-tetrahydrocannabinolic acid. A cannabichromenic acid synthase is used to convert cannabigerolic acid into cannabichromenic acid. These three olivetolic acid-derived compounds (i.e., cannabidiolic acid, Δ⁹-tetrahydrocannabinolic acid, and cannabichromenic acid) are themselves converted to even more diverse cannabinoids via a combination of oxidation, decarboxylation, and isomerization reactions, which can be catalyzed using either biological or synthetic catalysts, or can also occur spontaneously following heating and/or application of UV light. For example, cannabidiol results from cannabidiolic acid decarboxylation, Δ⁹-tetrahydrocannabinol results from Δ⁹-tetrahydrocannabinolic acid decarboxylation, and subsequent isomerization of Δ⁹-tetrahydrocannabinol results in Δ⁶-tetrahydrocannabinol.

The term “optional” or “optionally” as used herein mean that the subsequently described feature or structure may or may not be present, or that the subsequently described event or circumstance may or may not occur, and that the description includes embodiments where a particular feature or structure is present and embodiments where the feature or structure is absent, or embodiments where the event or circumstance occurs and embodiments where it does not.

A non limiting illustration of the cannabinoid pathway is included in the scheme below.

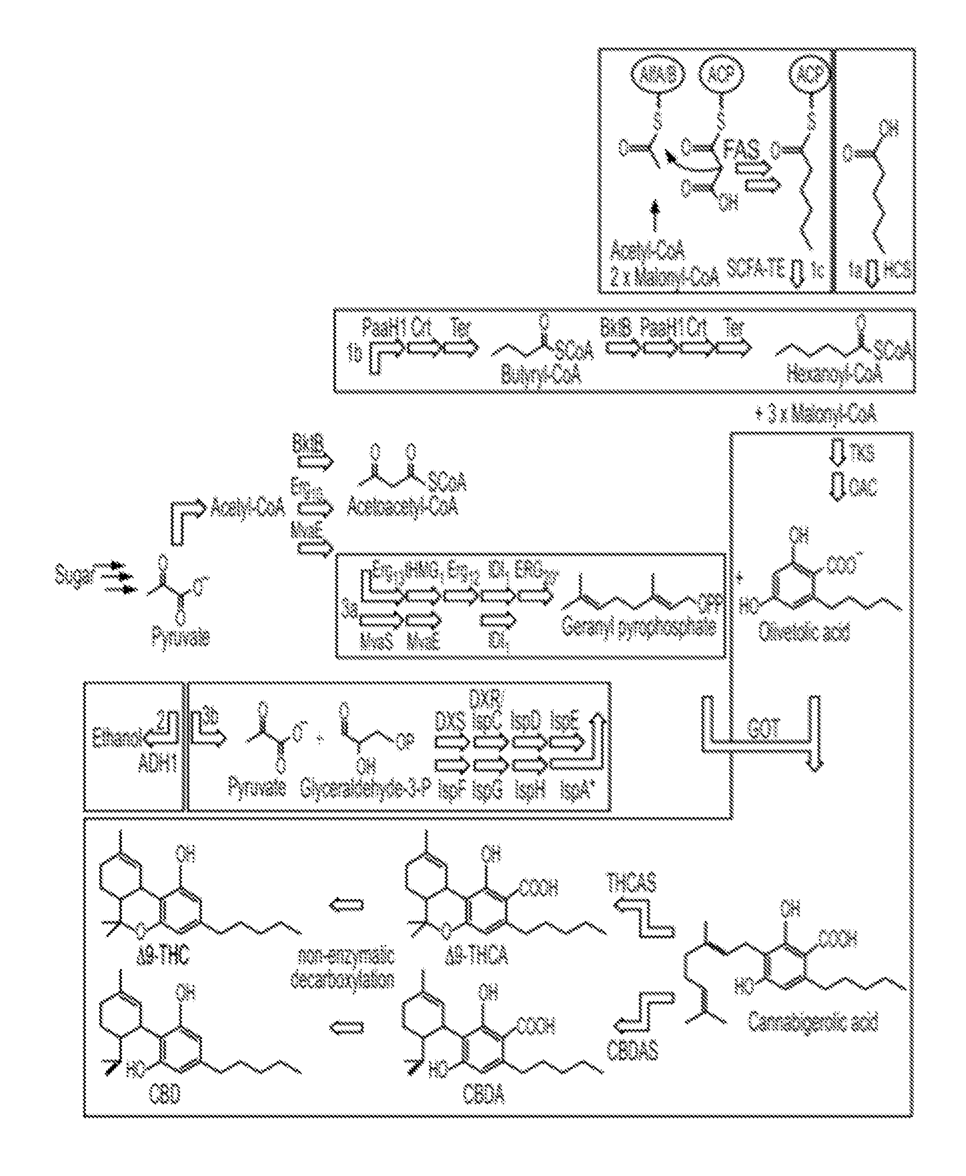

### Descriptive Embodiments

In one aspect, provided herein is a process for preparing one or more of a compound of formula (IA), (IB), and (IC):

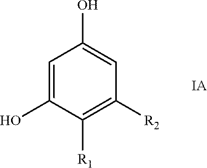

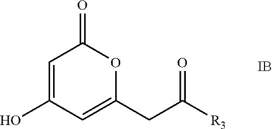

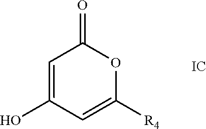

or a salt or an ester (carboxy and /or phenolic) thereof, wherein
R₁ is H or CO₂H;
each R₂, R₃, and R₄ is independently C₃-C₁₀ alkyl, C₃-C₁₀ alkenyl, or C₃-C₁₀ alkynyl, preferably, C₃-C₈ alkyl, more preferably, n-pentyl or n-propyl; the process comprising:
fermenting a recombinant microorganism comprising: a polyketide synthase, wherein the polyketide synthase combines an acyl-CoA and two or more, such as two or three, malonyl-CoA to produce a polyketide thereby preparing one or more of a compound of formula (IA), (IB), and (IC) or the salt or the ester thereof. Optionally a dimeric α+β barrel (DABB) protein is also co-expressed with the polyketide resulting in a polyketide comprising a carboxylic acid.

In one embodiment the ester is independently a carboxylic acid ester, or in other words, the carboxylic acid moiety corresponding to R¹ is esterified. In another embodiment, the ester is independently a phenolic ester.

In another embodiment, at least one compound prepared is of formula (IA). Without being bound by theory, the polyketide synthase combines an acyl-CoA and three malonyl-CoA to prepare a compound of formula IA, where R₁= H.

In another embodiment, at least one compound prepared is of formula (IB).

In another embodiment, at least one compound prepared is of formula (IC).

In another embodiment, one or more phenolic hydroxy moieties of the compound of formula (IA), (IB), or (IC), or a salt thereof is esterified in vivo (or endogenously) as a result of overexpression of an arylesterase in the microorganism.

In another embodiment, the compound of formula (IA), (IB), or (IC) is glycosylated in vivo as a result of overexpression of a glycosylase in the microorganism.

In another embodiment, the microorganism is a fungus. In another embodiment, the microorganism is a bacteria. In another embodiment, the microorganism is an algae. In another embodiment, the microorganism is yeast. In another embodiment, the microorganism is S. cerevisiae.

In some embodiments, the microorganism is a prokaryotic organism. In some embodiments, the microorganism is an eukaryotic organism. In some embodiments, the microorganism is a fungal organism. In some embodiments, the microorganism is a yeast organism. In some embodiments, the microorganism is a bacterial organism In some embodiments, the microorganism is a unicellular organism. In some embodiments, the microorganism is is a bacterial cell. In some embodiments, the microorganism is an eukaryote. In some embodiments, the microorganism is is a yeast cell. In various embodiments, the yeast is selected from the non-limiting list of example genera: Candida, Cryptococcus, Hansenula, Issatchenkia, Kluyveromyces, Komagataella, Lipomyces, Pichia, Rhodosporidium, Rhodotorula, Saccharomyces or Yarrowia. In some embodiments, the microorganism is is a fungus. In some embodiments, the microorganism is an algae. In some embodiments, the microorganism is a P. kudriavzevii organism. In some embodiments, the microorganism is a P. pastoris organism. In some embodiments, the microorganism is a S. cerevisiae organism. In some embodiments, the microorganism is a Y. lipolytica organism. In some embodiments, the microorganism is a Kluyveromyces marxianus organism.

In some embodiments, the microorganism is a bacterial cell. In some embodiments, the microorganism is a bacterial cell selected from Bacillus, Clostridium, Corynebacterium, Escherichia, Pseudomonas, and Streptomyces. In some embodiments, the microorganism is an E. coli organism.

As is apparent to the skilled artisan, the microorganisms disclosed herein are host cells for the purpose of this invention.

In another embodiment, the microorganism is fermented aerobically in the presence of a water immiscible, liquid, hydrophobic phase which dissolves the one or more of a compound of formula (IA), (IB), and (IC) or the salt or ester thereof. In another embodiment, the process further comprises separating the hydrophobic phase from an aqueous phase comprising the microorganism, the separating comprising a first continuous centrifugation to separate the cells and a bulk of a spent broth from the hydrophobic phase, followed by a second continuous centrifugation to separate the hydrophobic phase from the remaining aqueous phase. In another embodiment, the process further comprises: esterifying, isoprenylating, or performing an annulation of the compound included in the hydrophobic phase, under conditions suitable to perform an esterification, isoprenylation, or annulation without the need for a solvent swap. In another embodiment, the compound prepared is isoprenylated. In another embodiment, the compound prepared is esterified. In another embodiment, the compound prepared is made to undergo an annulation.

In certain embodiments, carbon feedstocks are utilized for production of olivetol or another compound produced herein. Suitable carbon sources include, without limitation, those selected from the group consisting of purified sugars (e.g., dextrose, sucrose, xylose, arabinose, lactose, etc.); plant-derived, mixed sugars (e.g., sugarcane, sweet sorghum, molasses, cornstarch, potato starch, beet sugar, wheat, etc.), plant oils, fatty acids, glycerol, cellulosic biomass, alginate, ethanol, carbon dioxide, methanol, and synthetic gas (“syn gas”).

In some embodiments, one or multiple intermediates and precursors of the cannabinoid pathways, including sugar, an acid of formula R₂-CO₂H or a salt thereof, malonic acid, hexanoic acid, mevalonate, olivetol, and olivetolic acid are employed as a feedstock. In some embodiments, an acid of formula R₂-CO₂H or a salt thereof is employed as a feedstock. In one embodiment the one or multiple intermediates and precursors of the cannabinoid pathway are utilized in a cell free system, e.g., and without limitation, with a prenyl transferase that condenses olivetol/olivetolic acid and GPP. In some embodiments, another enzyme of the cannabinoid pathway is incorporated in a host cell (or added to a cell free system) to further process CBGA or CBG into THCA, CBDA, THC, CBD or other CBG(A) derivative. Without being bound by theory, in some embodiments, such a feedstock would result in a commercially relevant process without the limitations and timelines associated with careful balancing of full pathway enzymes in a cell or cell-free system.

A given host cell may catabolize a particular feedstock efficiently or inefficiently. If a host cell inefficiently catabolizes a feedstock, then one can modify the host cell to enhance or create a catabolic pathway for that feedstock. Additional embodiments of the invention include the use of methanol catabolizing host strains. In some embodiments, the host is a yeast strain. In some embodiments, the host is selected from S. cerevisiae, Pichia kudriavzevii, Komagataella pastoris, Pichia methanolica, or Pichia pastoris.

The invention utilizes microorganisms and host cells comprising genetic modifications that increase titer, yield, and/or productivity of olivetol or another compound produced herein through the increased ability to catabolize non-native carbon sources. Wild type S. cerevisiae cells are unable to catabolize pentose sugars, lignocellulosic biomass, or alginate feedstocks. In some embodiments, the invention provides a S. cerevisiae cell comprising a heterologous nucleic acid encoding enzymes enabling catabolism of pentose sugars useful in production of olivetol, as described herein. In other embodiments, the heterologous nucleic acid encodes enzymes enabling catabolism of lignocellulosic feedstocks. In yet other embodiments of the invention, the heterologous nucleic acid encodes enzymes increasing catabolism of alginate feedstocks.

In another embodiment, the compound dissolved in the hydrophobic phase is one or both of olivetolic acid or a salt thereof and olivetol.

In another aspect, provided herein is a process comprising:
aerobically fermenting a recombinant microorganism comprising: a polyketide synthase, optionally an olivetolic acid cyclase (OAC), and further optionally a hexanoyl Co-A synthetase (HCS), wherein the fermenting is performed in the presence of a water immiscible, liquid, hydrophobic phase,
to prepare one or more of: olivetolic acid or a salt or ester thereof, and olivetol or an ester thereof,
wherein the hydrophobic phase dissolves olivetolic acid or a salt or ester thereof or olivetol or an ester thereof.

In another embodiment, the olivetolic acid is partially or completely esterified endogenously within the microorganism to prepare the olivetolic acid ester.

In another embodiment, the olivetolic acid ester is prepared exogenously comprising esterifying olivetolic acid with an alcohol under conditions suitable to prepare an olivetolic acid ester.

The esterification can be performed in presence of suitable esterification catalyst, as is well known to the skilled artisan. In some embodiments the esterification catalyst is soluble in the hydrophobic phase utilized herein, and partitions partially or completely into the hydrophobic phase.

In another embodiment, one or more hydroxyl moieties of olivetolic acid, olivetol, or an olivetolic acid ester is partially or completely glycosylated by the microorganism to provide glycosylated olivetolic acid, glycosylated olivetol, or glycosylated olivetolic acid ester. In some embodiments, the glycosylating microorganism overexpresses glycosylation enzymes. In some embodiments, the glycosylating microorganism overexpresses enzymes producing UDP-glucose.

In some embodiments, hexanoic acid or a salt of each thereof is exogenously supplied to a reactor where the fermenting occurs. In some embodiments, 3-oxooctanoic acid, or a salt of each thereof is exogenously supplied to a reactor where the fermenting occurs. In some embodiments, 3,5-dioxodecanoic acid or a salt of each thereof is exogenously supplied to a reactor where the fermenting occurs. In some embodiments, 3,5,7-trioxododecanoic acid or a salt of each thereof is exogenously supplied to a reactor where the fermenting occurs.

In one embodiment, the process further comprises separating the hydrophobic phase from an aqueous phase, the separating comprising a first continuous centrifugation to separate the cells and a bulk of a spent broth from the hydrophobic phase, followed by a second continuous centrifugation to separate the hydrophobic phase from the remaining aqueous phase. In one embodiment, the process further comprises isoprenylating the olivetol, olivetolic acid or a salt thereof, or the olivetolic acid ester included in the hydrophobic phase, without the need for a solvent swap, under conditions suitable to perform an isoprenylation, to prepare a cannabinoid or a mixture of cannabinoids.

In another embodiment, the olivetolic acid or the salt thereof contained in the hydrophobic phase is esterified with an alcohol under conditions suitable to esterify the carboxyl moiety of olivetolic acid or a salt thereof to yield alkyl olivetolate. In another embodiment, the alcohol is selected from alcohols with 2 or more carbons such as C₂-C₈ alcohols.

In another embodiment, the alkyl olivetolate is reacted with an isoprenoid, or is isoprenylated, to produce a cannabinoid. In some embodiments, the reaction is catalyzed by a Bronsted acid. In some embodiments, the reaction is catalyzed by a Lewis acid. Examples of suitable catalysts include without limitation organic acids (e.g. trifluoroacetic acid, methanesulfonic acid, tosic acid, and the likes), mineral acids or solutions of mineral acids (e.g. hydrochloric acid, nitric acid, sulfuric acid, and the likes), polymer-supported acids (e.g. Amberlyst-15, polymer-supported tosic acid, Lewis acids (e.g. BF₃, Sc(OTf)₃, and the likes), amino acids, or organocatalysts.

In another embodiment, the cannabinoid or the cannabinoid mixture comprises a carboxyl moiety or a salt thereof, and is decarboxylated under conditions suitable for decarboxylation, to prepare a decarboxylated cannabinoid. The decarboxylation can be modulated by heating the solution and/or by addition of a catalyst and/or by the addition of a base.

In another embodiment, the olivetolic acid or the salt thereof contained in the hydrophobic phase is decarboxylated under conditions suitable for decarboxylation to provide an initial composition comprising olivetol. An acid may be added before or during decarboxylation to modulate the decarboxylation reaction. The reaction mixture may be heated to increase the decarboxylation rate. A base may be added to modulate the decarboxylation reaction.

In another embodiment, the initial composition comprising olivetol is isoprenylated under conditions suitable for isoprenylating a phenolic compound. Examples of compounds useful in isoprenylating include without limitation geraniol, farnesol, geranylgeraniol, p-mentha-2,8-dien-1-ol (or (1S,4R)-p-Mentha-2,8-dien-1-ol), citral, and the likes. In some embodiments, the isoprenylated compound is cannabigerol (CBG). In another embodiment, the isoprenylated compound is cannabigerolic acid (CBGA).

In some embodiments, the initial composition comprising olivetol is reacted with geraniol and an acid under conditions suitable to undergo a Friedel-Crafts alkylation to provide cannabichromene. The reaction may be performed in a suitable solvent, e.g., a solvent that is inert to the reactants and the reagents. In some embodiments, a Bronsted acid is employed. In some embodiments, a Lewis acid is employed. The acid can be used in catalytic amounts. In some embodiments, the isoprenylated compound is cannabigerol (CBG). In some embodiments, the isoprenylated compound is cannabigerolic acid (CBGA). Suitable acids include a Bronsted acid such as a sulfonic acid, such as p-toluene sulfonic acid, or a Lewis acid such as BF₃ etherate, and the likes. Methods for reacting geraniol with olivetol are known in the art, which can be modified by the skilled artisan based on the present disclosure to provide cannabigerol as provided herein. See, e.g., J. Biol. Chem., Vol. 271, No. 29, Issue of July 19, pp. 17411-17416, 1996 (incorporated herein by reference). Unreacted olivetol may be separated by one or more or crystallization and chromatography.

In some embodiments, the initial composition comprising olivetol is reacted with citral under conditions suitable to undergo cyclization to provide cannabichromene (CBC). The reaction may be performed in a suitable solvent, e.g., a solvent that is inert to the reactants and the reagents. In some embodiments, the isoprenylated compound is cannabichromene (CBC). In some embodiments, the isoprenylated compound is cannabichromic acid (CBCA). In some embodiments, citral reacts with olivetol under basic conditions to provide cannabichromene. Suitable bases include a primary amine, such as without limitation propyl amine and tertiary butyl amine; pyridine; and the likes. Methods for reacting citral with olivetol are known in the art, which can be modified by the skilled artisan based on the present disclosure to provide cannabichromene as provided herein. See, e.g., J. Heterocyclic Chem., volume15, Issue 4,1978, pages 699-700 and U.S. Pat. No. 4,315,862 (each incorporated herein by reference). Unreacted olivetol may be separated by washing with alkali such as NaOH and the likes, or by chromatography with alkali impregnated silica.

In another embodiment, the cannabinoid composition is purified, optionally hydrolyzed, and isolated to provide one or more cannabinoids Hydrolysis may precede or follow purification. In another embodiment, the total cannabinoids contained in the isolated product is at least 25%, or 50%, or 75%, or 90%, or 95%, or 98%, or 99% of a single cannabinoid. In some embodiments, after purification, the remaining amount may include one or more of a different regioisomer, a different enantiomer, a different diastereomer, or a solvent

In another embodiment, the cannabinoid is cannabigerolic acid (CBGA). In another embodiment, the cannabinoid is cannabichromenic acid (CBCA). In another embodiment, the cannabinoid is cannabinolic acid (CBNA). In another embodiment, the cannabinoid is tetrahydrocannabinoic acid (THCA). In another embodiment, the cannabinoid is cannabidiolic acid (CBDA). In another embodiment, the cannabinoid is cannabigerol (CBG). In another embodiment, the cannabinoid is cannabichromene (CBC). In another embodiment, the cannabinoid is cannabinol (CBN). In another embodiment, the cannabinoid is tetrahydrocannabinol (THC). In another embodiment, the cannabinoid is cannabidiol (CBD). In another embodiment, the cannabinoid is a prenylogous version of the above (e.g. sesqui-CBG). In another embodiment, the cannabinoid is a compound that causes activation of the CB1, CB2, or TRP receptor.

In another aspect, provided herein is a process comprising:
aerobically fermenting a recombinant microorganism comprising: a polyketide synthase, optionally an olivetolic acid cyclase (OAC), and further optionally butyryl Co-A synthetase, wherein the fermenting is performed in the presence of a water immiscible, liquid, hydrophobic phase;
to prepare one or more of: divarinic acid or a salt or ester thereof, and divarin,
wherein the hydrophobic phase dissolves divarinic acid or a salt or ester thereof or divarin, as they are prepared.

In one embodiment, the divarinic acid is partially or completely esterified endogenously within the microorganism to prepare the divarinic acid ester. The OH and/or the CO₂H can be esterfied.

In another embodiment, the divarinic acid ester is prepared exogenously comprising esterifying olivetolic acid with an alcohol under conditions suitable to esterify a carboxylic acid.

The esterification can be performed in presence of suitable esterification catalyst, as is well known to the skilled artisan. In some embodiments the esterification catalyst is soluble in the hydrophobic phase utilized herein, and partitions partially or completely into the hydrophobic phase.

In another embodiment, one or more hydroxyl moieties of divarinic acid, divarin, or divarinate esters are partially or completely glycosylated by the microorganism to provide glycosylated divarinic acid or a salt thereof, glycosylated divarin, or glycosylated divarinate ester. In some embodiments, the glycosylating microorganism overexpresses glycosylation enzymes. In some embodiments, the glycosylating microorganism overexpresses enzymes producing UDP-glucose.

In some embodiments, butyric acid or a salt of each thereof is exogenously supplied to a reactor where the fermenting occurs. In some embodiments, 3-oxooctanoic acid, or a salt of each thereof is exogenously supplied to a reactor where the fermenting occurs. In some embodiments, 3,5-dioxodecanoic acid or a salt of each thereof is exogenously supplied to a reactor where the fermenting occurs. In some embodiments, 3,5,7-trioxododecanoic acid or a salt of each thereof is exogenously supplied to a reactor where the fermenting occurs.

In another embodiment, the process further comprising separating the hydrophobic phase from an aqueous phase, the separating comprising a first continuous centrifugation to separate the cells and the bulk of the spent broth from the hydrophobic phase, followed by a second continuous centrifugation to separate the hydrophobic phase from the remaining aqueous phase.

In another embodiment, the process further comprises isoprenylating the divarin, divarinic acid, or the divarinic acid acid ester included in the hydrophobic phase, without the need for a solvent swap, under conditions suitable to perform an isoprenylation, to prepare a cannabinoid or a mixture of cannabinoids.

In another embodiment, the divarinic acid or the salt thereof contained in the hydrophobic phase is esterified with an alcohol under conditions suitable for esterification to provide alkyl divarinate.

In another embodiment, the alcohol utilized for esterification is selected from alcohols with 2 or more carbons such as C₂-C₈ alcohols.

In another embodiment, the alkyl divarinate is reacted with an isoprenoid, or is isoprenylated, to produce a cannabinoid. In some embodiments, the reaction is catalyzed by a Bronsted acid. In some embodiments, the reaction is catalyzed by a Lewis acid. Examples of suitable catalysts include without limitation organic acids (e.g. trifluoroacetic acid, methanesulfonic acid, tosic acid, and the likes), mineral acids or solutions of mineral acids (e.g. hydrochloric acid, nitric acid, sulfuric acid, and the likes), polymer-supported acids (e.g. Amberlyst-15, polymer-supported tosic acid, Lewis acids (e.g. BF₃, Sc(OTf)₃, and the likes), amino acids, or organocatalysts.

In another embodiment, the cannabinoid or the cannabinoid mixture comprises a carboxyl moiety or a salt thereof, and is decarboxylated under conditions suitable for decarboxylation, to prepare a decarboxylated cannabinoid. The decarboxylation can be modulated by heating the solution and/or by addition of a catalyst and/or by the addition of a base.

In another embodiment, the divarinic acid or the salt thereof contained in the hydrophobic phase is decarboxylated to provide an initial composition comprising divarin. An acid may be added before or during decarboxylation to modulate the decarboxylation reaction. The reaction mixture may be heated to increase the decarboxylation rate. A base may be added to modulate the decarboxylation reaction.

In another embodiment, the initial composition comprising divarin is isoprenylated under conditions suitable for isoprenylating a phenolic compound. Examples of compounds useful in isoprenylating include without limitation geraniol, farnesol, geranylgeraniol, p-mentha-2,8-dien-1-ol ((1S,4R)-p-Mentha-2,8-dien-1-ol), citral, and the likes. In another embodiment, the isoprenylated compound is cannabigerovarin. In another embodiment, the isoprenylated compound is cannabigerovarinic acid.

In some embodiments, the initial composition comprising divarin is reacted with geraniol and an acid under conditions suitable to undergo a Friedel Crafts alkylation to provide cannabigerovarin (CBGV). The reaction may be performed in a suitable solvent, e.g., one that is inert to the reactants and the reagents. In some embodiments, a Bronsted acid is employed. In some embodiments, a Lewis acid is employed. The acid can be used in catalytic amounts. In some embodiments, the isoprenylated compound is cannabigerovarin (CBGV). In some embodiments, the isoprenylated compound is cannabigerovarinic acid (CBGVA). Suitable acids include a sulfonic acid, such as p-toluene sulfonic acid, BF₃ etherate, and the likes. A skilled artisan will be able to adapt and modify, in view of this disclosure, known processes for preparing CBG and CBGA for preparing CBGV and CBGVA.

In some embodiments, the initial composition comprising divarin is reacted with citral under conditions suitable to undergo cyclization to provide cannabichromevarin (CBCV). In some embodiments, the isoprenylated compound is cannabichromevarin (CBCV). In some embodiments, the isoprenylated compound is cannabichromevarinic acid (CBCVA). In some embodiments, citral reacts with divarin under basic conditions to provide cannabichromevarin. Suitable bases and other conditions will be apparent to the skilled artisan upon reading this disclosure. Unreacted divarin can be separated by reacting with alkali.

In another embodiment, the cannabinoid composition is purified, optionally hydrolyzed, and isolated to yield one or more cannabinoids. In another embodiment, the total cannabinoids contained in the isolated product is at least 25%, or 50%, or 75%, or 90%, or 95%, or 98%, or 99% of a single cannabinoid.

In another embodiment, the cannabinoid is cannabigerovarinic acid (CBGVA). In another embodiment, the cannabinoid is cannabichromevarinic acid (CBCVA). In another embodiment, the cannabinoid is cannabinovarinic acid (CBNVA). In another embodiment, the cannabinoid is tetrahydrocannabivarinic acid (THCVA). In another embodiment, the cannabinoid is cannabidivarinic acid (CBDVA). In another embodiment, the cannabinoid is cannabigerovarin (CBGV). In another embodiment, the cannabinoid is cannabichromevarin (CBCV). In another embodiment, the cannabinoid is cannabivarin (CBNV). In another embodiment, the cannabinoid is tetrahydrocannabivarin (THCV). In another embodiment, the cannabinoid is cannabidivarin (CBDV). In another embodiment, the cannabinoid is meroterpenoid compound that causes activation of the CB1, CB2, or TRP receptors.

In another embodiment, the illustrative and nonlimiting examples of an acyl-CoA includes Oleoyl-CoA, Palmitoleoyl-CoA, Stearoyl-CoA, Dehydrostearoyl-CoA, Oxostearoyl-CoA, Enoyl-CoA, Oxacyl-CoA, Hexanoyl-CoA, Oxohexanoyl-CoA, Butanoyl (or Butyryl)-CoA, Crotonoyl-CoA, Acetoacetyl-CoA, Pentanoyl-CoA, or Oxopentanoyl-CoA.

In one embodiment, the acyl-CoA is a synthetic molecule that functions similar to an acyl-CoA and is accepted by the polyketide synthase enzyme. Non limiting examples of such synthetic molecules are provided, e.g., in Prasad, Gitanjeli et al. “A mechanism-based fluorescence transfer assay for examining ketosynthase selectivity.” Organic & biomolecular chemistry vol. 10,33 (2012): 6717-23. Incorporated herein by reference.

In another embodiment, the polyketide synthase is olivetol synthase (OLS) having an amino acid sequence that is at least 95%, at least 96%, at least 97%, at least 98%, or at least 99% identical with SEQ ID 1. In another embodiment, the polyketide synthase shares at least 50% sequence identity with the amino acid sequence of SEQ ID 1 and whose alpha carbon backbone of its structure does not deviate by more than 1.5 Å with (OLS) having the amino acid sequence of SEQ ID 1.

In another embodiment, the DABB protein is olivetolic acid cyclase (OAC) having an amino acid sequence that is at least 95%, at least 96%, at least 97%, at least 98%, or at least 99% identical with SEQ ID 2. In another embodiment, the DABB protein has an amino acid sequence that is at least at least 50% identical to olivetolic acid cyclase (OAC) of SEQ ID 2. In another embodiment, the OAC has an amino acid sequence at least 95%, at least 96%, at least 97%, at least 98%, or at least 99% identical with SEQ ID 4, which is described below.

Sequence ID 2: OAC/DABBMAVKHLIVLKFKDEITEAQKEEFFKTY
VNLVNIIPAMKDVYWGKDVTQKKEEGYTHIVEVTFESV ETIQD YIIHP
AHVGF GOVYR SFWEK LLIFD YTPRK

In another embodiment, the microorganism comprises an acyl-CoA synthetase enzyme. Acyl-CoA refers, as is well known, to an ester of coenzyme A with a carboxylic acid. An acyl-CoA synthetase enzyme converts a carboxylic acid to an acyl-CoA. In another embodiment, the microorganism comprises an acyl-CoA synthetase enzyme, which is CsAAE1 having an amino acid sequence of SEQ ID 3. In another embodiment, the microorganism comprises an acyl-CoA synthetase enzyme having an amino acid sequence that is at least 50-75% identical with the amino acid sequence of SEQ ID 3. In another embodiment, at least a part of the acyl-CoA or a salt thereof is exogenously added to a reactor where the fermenting occurs. In another embodiment, the acyl-CoA like synthetic substrate or a salt thereof is exogenously added to a reactor where the fermenting occurs. In another embodiment, the carboxylic acid corresponding to the acyl-CoA or a salt thereof is exogenously added to a reactor where the fermenting occurs.

Sequence ID 3: CsAAE1MGKNYKSLDSVVASDFIALGITSEVAETL
HGRLAEIVCNYGAATPQTWINIANHILSPDLPFSLHQMLFYGCYKDFGPA
PPAWIPDPEKVKSTNLGALLEKRGKEFLGVKYKDPISSFSHFQEFSVRNP
EVYWRTVLMDEMKISFSKDPECILRRDDINNPGGSEWLPGGYLNSAKNCL
NVNSNKKLNDTMIVWRDEGNDDLPLNKLTLDQLRKRVWLVGYALEEMGLE
KGCAIAIDMPMHVDAWIYLAIVLAGYVWSIADSFSAPEISTRLRLSKAKA
IFTQDHIIRGKKRIPLYSRVVEAKSPMAIVIPCSGSNIGAELRDGDISWD
YFLERAKEFKNCEFTAREQPVDAYTNILFSSGTTGEPKAIPWTQATPLKA
AADGWSHLDIRKGDVIVWPTNLGWMMGPWLVYASLLNGASIALYNGSPLV
SGFAKFVQDAKVTMLGVVPSIVRSWKSTNCVSGYDWSTIRCFSSSGEASN
VDEYLWLMGRANYKPVIEMCGGTEIGGAFSAGSFLQAQSLSSFSSQCMGC
TLYILDKNGYPMPKNKPGIGELALGPVMFGASKTLLNGNHHDVYFKGMPT
LNGEVLRRHGDIFELTSNGYYHAHGRADDTMNIGGIKISSIEIERVCNEV
DDRVFETTAIGVPPLGGGPEQLVIFFVLKDSNDTTIDLNOLRLSFNLGLQ
KKLNPLFKVTRVVPLSSLPRTATNKIMRRVLRQQFSHFE

In one embodiment, the hydrophobic phase utilized herein comprises an alkane. In one embodiment, the hydrophobic phase comprises an alcohol preferably with carbon number greater than 4 such as a C₅-C₈ alcohol. In one embodiment, the hydrophobic phase comprises an ester. In one embodiment, the hydrophobic phase comprises a triglyceride. In one embodiment, the hydrophobic phase comprises a diester such as dialkyl malonate. In one embodiment, the hydrophobic phase comprises a commercially available oil. Examples of such oils include without limitation sunflower oil, olive oil, vegetable oil or the like). In one embodiment, the hydrophobic phase comprises a combination of the various hydrophobic phases disclosed hereinabove.

Methods for prenylating olivetol, olivetolic acid, olivetolc acid esters, divarin, divarinic acid, divarinic acid esters, and such other resorcinol derivatives utilized herein, e.g., and without limitation, with geraniol, citral, cyclic isoprenoids, and the likes are known in the art (see e.g., WO2019033168, US2017/283837, US2015/336874, US2018/244642, US2009/36523, WO2010/59943, US4315862, each of which is incorporated herein in its entirety by reference), and can be modified based on the disclosure provided herein by a skilled artisan.

The biosynthesis of certain illustrative and nonlimiting cannabinoids, as utilized herein, is described below.

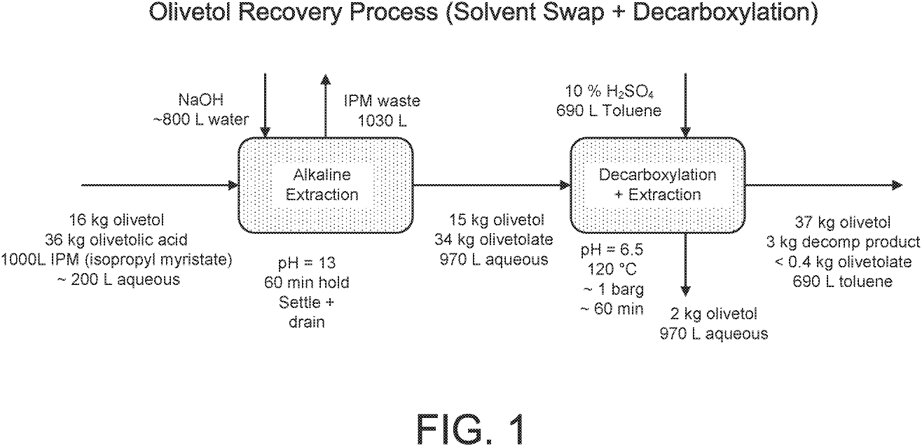

The scheme below illustrates aromatic polyketides (1A), ketones (FIG. 1B) and lactones (FIG. 1C) and other diverse class of chemical compounds that have a wide range of applications in the industrial and.

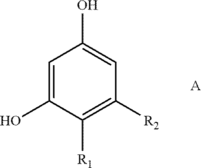

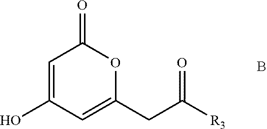

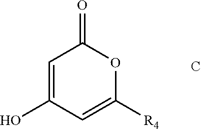

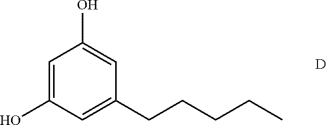 [SMILES: CCCCCc1cc(O)cc(O)c1C(=O)O]

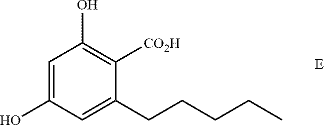

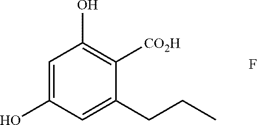

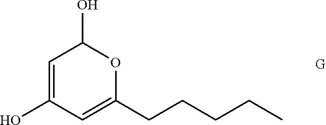 [SMILES: CCCCCC(=O)Cc1cc(O)cc(=O)o1]

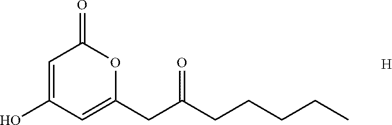

General examples of the production of acid or non-acidic polyketides (IA), the production of ketones (IB), and the production of lactones (IC). Specific examples are the production of the polyketide olivetol (ID), olivetolic acid (IE), the lactone PDAL (IG) and a ketone HTAL (IH). For example, and not for limitation, all products are produced using a polyketide synthase olivetol synthase (OLS); other suitable TKS enzymes are also useful. To make olivetolic acid a DABB protein, here olivetolic acid cyclase (OAC) is co-expressed with the polyketide synthase to produce the acidic polyketide. ID, IE, IG, and IH are formed, for example, from malonyl-CoA and hexanoyl-CoA while F is form from malonyl-CoA and butyl-CoA.

In one embodiment, a compound of formula ID is provided. In one embodiment, a compound of formula IE or a salt thereof is provided. In one embodiment, a compound of formula IF or a salt thereof is provided. In one embodiment, a compound of formula IG is provided. In one embodiment, a compound of formula IH is provided.

In some embodiments, provided herein are biosynthetic cannabinoids, as distinct from cannabinoids made by chemical synthesis only, where such biosynthetic cannabinoids comprise trace or tell-tale amounts (less than 3%, preferably less than 2%, more preferably less than 1 %) of fermentation derived byproducts. In some embodiments, the byproduct is a lactone of formula IC, such as PDAL.

These compounds are produced by many natural sources and represent a valuable class of natural products. In nature many different plants and microorganisms produce these types of compounds. These compounds can be functional molecules themselves or are used to created more complex compounds through additional chemical steps such as prenylation or esterification. In nature aromatic polyketides, ketones, and lactones are formed from the combination of 2 or more malonyl-CoA molecules and also typically involve an additional substrate such as an acyl-CoA molecule. Herein we disclose a method to produce a diverse set of polyketides, ketones, and lactones using engineered microorganisms. We also describe a method wherein these compounds can be extracted and further processed to create additional molecules of value such as cannabinoids.

The invention described below focuses on the production of aromatic polyketides, ketones and lactones from yeast, bacteria, and/or algae. The invention can be used to create diverse chemical libraries for therapeutic screening or as an industrial scale production platform for high value natural compounds. In one aspect of the invention diverse chemical compounds can be form by supplementing the media surrounding engineered cells with different acyl-CoA compounds or different acyl-acid compounds which are then transformed into acyl-CoA compounds in vivo. In another aspect of the invention the aromatic polyketides, ketones and/or lactones can be extracted from the media using an immiscible hydrophobic layer that is added to the fermentation vesicle. In another aspect of the invention these aromatic polyketides, ketones and/or lactones, once produced and collected, can be used to create more complex chemical components through the chemical reactions such as but not limited to prenylation or esterification.

In another embodiment, the OAC utilized herein has Sequence ID 4:

MAVKHLIVLKFKDEITEAQKEEFFKTYVNLVNIIPAMKDVYWGKDVTQKN
KEEGYTHIVEVTFESVETIQDYIIHPAHVGFGDVYRSFWEKLLIFDYTPR
K

### In Vivo Production of Aromatic Polyketides, Ketones And/Or Lactones

The aromatic polyketides, ketones and lactones of interest are formed from fatty chain acyl-CoA and condensation of malonyl-CoA. In order to produce these compounds, the enzyme that condense these compounds together (a polyketide synthase) must take at least 2 malonyl-CoA. There are many different polyketide synthase enzymes that perform this function. The choice of enzyme used must be able to be expressed in the host cell and function appropriately. One enzyme in particular, the polyketide synthase from the cannabis plant (OLS SEQ ID 1), can be used to create these different compounds. In this invention this enzyme has been functionally expressed into yeast in order to produce various polyketide products. This enzyme can also be expressed in bacteria and used in vitro to produce various polyketide, ketone, or lactone products. The same enzyme can be used to create either a lactone, ketone, or polyketide with the different between the final products having to do with the number of malonyl-CoAs involved and if/or when the product is hydrolyzed off. Lactones require 2 malonyl-CoAs while the ketones and polyketides required 3 malonyl-CoAs. Controlling what compound is formed can be done through engineering of the polyketide synthase enzyme, limiting availability of malonyl-CoA, or increasing the speed of hydrolysis. In some embodiments of this invention increasing or decreasing the availability of malonyl-CoA can lead to different product formations.

In addition to requiring malonyl-CoA the enzyme can also use acyl-CoA as substrates which leads to a diversity in the chemical products. There are several aryl-CoAs that act as substrates for the enzyme including but not limited to: Oleoyl-CoA, Palmitoleoly-CoA, Stearoyl-CoA, Dehydrostearoly-CoA, Oxostearoyl-CoA, Enoyl-CoA, Oxacyl-CoA, Hexanoyl-CoA, Oxohexanoyl-CoA, Butanoyl-CoA, Crotonoyl-CoA, Acetoacetyl-CoA, Pentanoyl-CoA, Oxopentanoyl-CoA. Synthetic molecules that have the same function as a CoA could also be used as substrates leading to increased chemical diversity. There are several ways in which various CoAs can be made. The production of fatty chain acyl-CoA or other specialized CoA containing compounds can be initiated in a variety of ways. In one embodiment enzymes can be introduced and overexpress to produce these compounds directly from sugar. There are several examples of the enzymes that are responsible for the production of fatty chain acyl-CoA are. In an alternative approach, fatty chain acyl-CoA can be produced by supplementing the growth media with a fatty acid and then incorporating a CoA charging enzyme. There are several examples of enzyme that can produce these types of CoAs including CsAAE1 (SEQ ID 2). Once the -CoA compound is formed it can be acted on by the polyketide synthase which results in different products being form.

In another aspect of the invention acidic polyketides can be made. These acidic polyketides can have unique therapeutic properties, such as novel antibiotics. In many cases the terminations and release of the polyketide product from the polyketide synthase results in its decarboxylation. In one aspect of this invention mixtures of acidic and non-acidic polyketides can be made by including an additional dimeric α+β barrel (DABB) protein. When this DABB protein is co-expressed with the polyketide synthase the resulting polyketide will have a carboxylic acid group. In one embodiment of this invention the dimeric α+β barrel (DABB) protein is the olivetolic acid cyclase enzyme which is found in the cannabis plant (OAC SEQ ID 3).

### Extraction of Aromatic Polyketides, Ketones and Lactones

In one aspect of the invention the extraction of the aromatic polyketides, ketones and/or lactones can be done In situ through the use of an immiscible organic solvent or oil layer. In order to achieve high product titers, the microorganism that is expressing the enzymes to produce the aromatic polyketides, ketones and/or lactones is made to secrete the products to the media; secretion can lead to high product titers. Often these products are not very water soluble or are toxic to the microorganisms themselves. Real-time removal of the product eliminates this toxicity as well as adds in streamlining further downstream processing. The choice of oil must have the following properties: the aromatic polyketides, ketones and lactones (the products) are soluble in this layer, the layer is immiscible with water, the layer is not toxic to the microorganism itself. Examples of useful extractions layers are dodecane and isopropyl myristate. Ideally the oil layer chosen has a high solubility for the products of interest and a low solubility for various media components such as sugars and vitamins to minimize additional downstream purification.

During fermentation or cell growth the oil layer is added to the growth vesicle. The compounds of interest that are produced are excreted from the cells and then collected in the immiscible oil layer creating a chemical sink for the product. This procedure extracts the compounds as well as minimizes their toxicity to the cells in solution. After fermentation has concluded the oil is separated from the growth media. There are several ways to separate the oil from the media and one such method would be centrifugation. In this example the oil is separated from the media by centrifugation, the oil is collected which contains the compounds of interest.

### Modification of Aromatic Polyketides, Ketones and Lactones

In one aspect of this invention after the aromatic polyketides, ketones or lactones are produced by the microorganism and are collected in the oil layer subsequent chemical reactions on the aromatic polyketides, ketones or lactones can occur. Additional modifications can be the dimerization of acidic polyketides, the esterification of acidic polyketides or the prenylation of polyketides, ketones or lactones or a combination of these chemical transformations through chemical or enzymatic means. Prenylation of the aromatic ring can lead to additional compounds that have the polyketide, lactone or ketone as their backbone. For the chemical synthesis of either prenylated products or other products the preferred reaction is with a molecule that contains a hydroxy group. Examples of these types of prenyl groups that could be attached to the aromatic ring would be farnesol, geraniol, prenol, citronellol, or 2-Methyl-3-buten-2-ol. For example, the addition of geraniol to olivetol creates cannabigerol (CBG), which is a natural cannabinoid. Other compounds can be added to the products derived from fermentation such as (1S,4R)-p-Mentha-2,8-dien-1-ol which results in the formation of cannabidiol or tetrahydrocannabidiol like molecules.

In some aspects of this invention it is preferable to use an oil layer that is compatible with subsequent chemical reactions. The choice of the oil used should follow the criterion described above and it is also preferable to choose an oil that allows for chemical reactions to take place. In some aspect of the invention the choice of oil is not compatible with additional chemical reactions. In this case the compounds must first be extracted from the oil layer and then reconstituted into a solvent that will allow for further chemical manipulations.

An illustrative and non-limiting process of isolating olivetol or another aromatic polyketide is schematically illustrated in FIG. 1.

In one embodiment, a mixture of compounds of an aromatic polyketide, the polyketide carboxylic acid, or a salt thereof provided by fermentation is extracted from a fermentation media by alkaline extraction. In some embodiments, the alkaline extraction is an aqueous alkaline extraction. In some embodiments, the alkaline extraction is performed at a pH of about 12 - about 14. In some embodiments, the alkaline extraction is performed at a pH of about 13.

In some embodiments, the extracted mixture of compounds of an aromatic polyketide, the polyketide carboxylic acid, or a salt thereof are decarboxylated to provide the aromatic polyketide. In some embodiments, the decarboxylation is performed by heating. In some embodiments, the heating is performed at about 100° C. - about 140° C., or preferably at about 110° C. - about 130° C. In some embodiments, the heating is performed at about 120° C. Post decarboxylation, the aromatic polyketide provided, comprises by weight about 2% or less, or preferably about 1% or less of the polyketide carboxylic acid, or a salt thereof. In some embodiments, the extracted mixture of an aromatic polyketide, the polyketide carboxylic acid, or a salt thereof are acidified before decarboxylation. In some embodiments, the decarboxylation is performed at a pH of about 5 - about 8. In some embodiments, the decarboxylation is performed at a pH of about 6.5.

In one embodiment, aromatic polyketide provided by decarboxylation is extracted into an organic solvent (e.g., a water immiscible organic solvent) to provide a solution of the compound of formula IA in the organic solvent. In some embodiments, the organic solvent is a solvent capable of dissolving a compound of the aromatic polyketide; the aromatic polyketide comprising an aromatic ring and polar hydroxy groups. In one embodiment, the organic solvent comprises an aromatic hydrocarbon solvent. In one embodiment, the organic solvent comprises toluene. In one embodiment, the organic solvent is toluene. In some embodiments, the organic solvent comprises aliphatic or alicyclic hydrocarbon solvents.

In some embodiments, the aromatic polyketide, present as a solution in the organic solvent, is reacted with a terpene alcohol, a terpenal (i.e., a terpene aldehyde), and the likes. In some embodiments, the solution of the aromatic polyketide in the organic solvent is employed for reacting the aromatic polyketide with a terpene alcohol. In some embodiments, the solution of the aromatic polyketide in the organic solvent is employed for reacting the compound the aromatic polyketide with a terpenal. In one embodiment, the terpine alcohol is geraniol. In one embodiment, the terpene alcohol is farnesol. In one embodiment, the terpene alcohol is menthadienol (trans 2,8-menthadienol or (1S,4R)-p-Mentha-2,8-dien-1-ol). In one embodiment, the terpene alcohol is trans 2,8-menthadienol. In one embodiment, the terpene alcohol is (1S,4R)-p-Mentha-2,8-dien-1-ol). In one embodiment, the terpenal is citral. In some embodiments, the reaction with a terpenal further comprises a primary amine. In one embodiment, the primary amine is tertiary butyl amine.

In some embodiments, the reaction of the aromatic polyketide with a terpene alcohol, a terpenal, or the likes provides a cannabinoid. In one embodiment, the cannabinoid is cannabigerol (CBG). In another embodiment, the cannabinoid is cannabichromene (CBC). In another embodiment, the cannabinoid is cannabidiol (CBD). In another embodiment, the cannabinoid is tetrahydrocannabinol (THC). In another embodiment, the cannabinoid is cannabinol (CBN). In another embodiment, the cannabinoid is the varin analog (CBGV, CBCV, CBDV, THCV, CBNV) of CBG, CBC, CBD, THC, CBN. A varin analog is a compound where the n-pentyl chain of a cannabinoid, e.g., and without limitation, CBG, CBC, CBD, or THC is replaced by an n-propyl chain. The cannabinoids obtained are purified by a variety of purification methods. In one embodiment, the purification method comprises chromatography. In one embodiment the purification method comprises distillation. In one embodiment, the chromatography comprises a reverse phase chromatography.

In one embodiment, the aromatic polyketide is olivetol. In another embodiment, the aromatic polyketide is divarin.

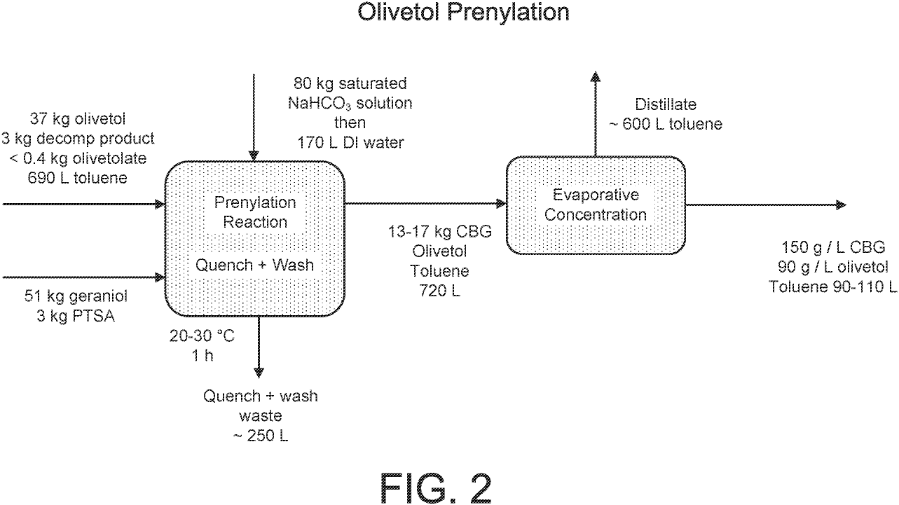

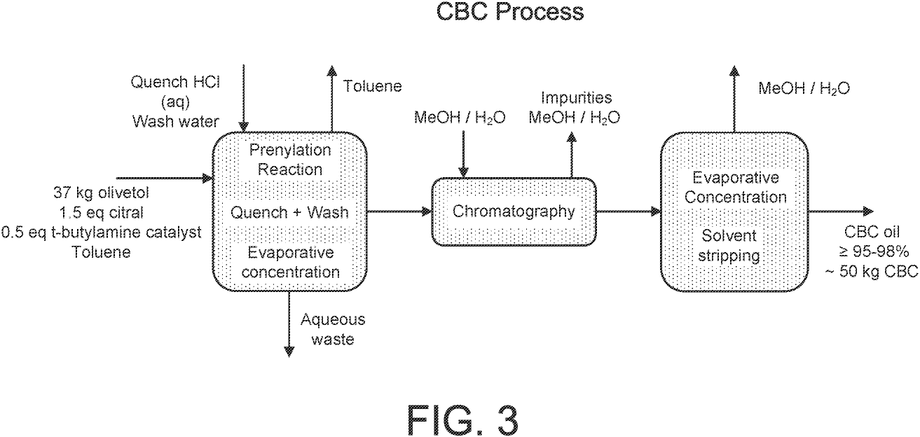

A non-limiting example of reacting (prenylating) olivetol with the terpene alcohol, geraniol, is schematically illustrated in FIG. 2. A non-limiting example of reacting (prenylating) olivetol with the terpenal, citral, is schematically illustrated in FIG. 3.

### EXAMPLES

These illustrative and non-limiting examples can be adapted according to the present disclosure to provide the methods and compositions provided herein.

### Example I Preparation of Cannabichromene

To a three-necked round bottomed flask (100 ml capacity), fitted with a dropping funnel and a condenser is added 5 g olivetol (27.8 mmole) and 2.03 g (2.96 ml, 27.8 mmole) t-butyl amine in 55 ml toluene and the mixture is heated to 50°-60° C., 4.23 g (4.76 ml, 27.8 mmole) of citral is then added dropwise. The mixture is refluxed for 9 hours, after which time it is cooled to room temperature and the solvent evaporated to give a crude reaction mixture.

### Example II Purification of Cannabichromene

5 g of the crude reaction mixture from Example I is dissolved in 100 ml toluene and the solution extracted twice with 50 ml of 1% aqueous sodium hydroxide solution followed by 50 ml of water. The toluene solution is then dried over anhydrous sodium sulfate and the solvent evaporated. The residue is then dissolved in 50 ml ethanol, and 250 mg of sodium borohydride are added portion-wise while stirring. Stirring at room temperature is continued for 30 minutes after which time the solvent is evaporated and the residue partitioned between water (50 ml.) and toluene (100 ml). The crude reaction mixture is chromatographed on a column of processed silica gel (200 g). Processed silica gel is prepared by making a paste of silica gel -PF254 with water (equal amount) which is then dried in an oven at 110° C. and the resulting cake passed through 60 mesh sieve. The solvent system used is a mixture of toluene and chloroform (1:1). Fractions are collected and the solvent evaporated to provide pure CBC.

Olivetol utilized in the examples provided herein can be replaced by other alkyl resorcinols, such as, 5-propyl-1,3-dihydroxybenzene or divarin to prepare “varin” and such other analogs of CBC such as CBCV and CBCVA. Citral utilized in the examples provided herein can be replaced by other terpene aldehydes such as farnesal to prepare isoprene homologs of CBC.

### Example III Preparation of Cannabiqerol (CBG)

Olivetol (2 g) and geraniol (3 g) are dissolved in 400 ml of chloroform containing p-toluenesulfonic acid (80 mg) and stirred at room temperature for 12 h in the dark. Chloroform may be replaced by toluene, cyclohexane, and such other solvents. The reaction mixture is washed with 400 ml of saturated sodium bicarbonate and then with 400 ml of water. After the chloroform layer is concentrated at 40° C. under reduced pressure, the residue is chromatographed on a 2.0 × 25-cm column of silica gel. The column is eluted with 1000 ml of toluene to give CBG (1.4 g).

Olivetol utilized in the examples provided herein can be replaced by other alkyl resorcinols, such as, 3-propyl-1,5-dihydroxybenzene to prepare “varin” and such other analogs of CBG. Geraniol utilized in the examples provided herein can be replaced by other terpenols such as farnesol to prepare isoprene homologs of CBG.

### Example IV Production of Cannabiqerol (CBG)-10L Scale

Olivetol (335 g) and geraniol (574 g) are dissolved in 5,500 g of toluene containing p-toluenesulfonic acid monohydrate (42.5 g) and stirred at 30° C. for 1.5 hr in a 10 L jacketed reactor. The reaction mixture is quenched with 700 ml of saturated sodium bicarbonate. After 30 minutes, agitation is stopped to allow phase separation. The aqueous layer is separated and discarded as waste. The organic layer is then washed with 2.7 L of DI-water for 30 minutes. After draining the aqueous layer, the organic layer is concentrated at 50° C. under reduced pressure to 150 g/L of CBG. The residue is chromatographed on a spherical silica gel column with a particle size distribution of 40-75 µm. The column is eluted with toluene and ethyl acetate gradient to purify the CBG from other impurities. A typical gradient is as follow:

| Step | Ethyl Acetate Start | Ethyl Acetate End | Length |
|---|---|---|---|
| Equilibration | 0% | 0% | 0.20 CV |
| 1 | 0% | 0% | 1.00 CV |
| 2 | 0% | 20% | 2.00 CV |
| 3 | 20% | 20% | 2.00 CV |
| 4 | 20% | 80% | 1.00 CV |

Toluene can be replaced by hexane, heptane, and such other solvents. Ethyl acetate can be replaced by 2-propanol or acetone. Olivetol utilized in the examples provided herein can be replaced by other alkyl resorcinols, such as, 3-propyl-1,5-dihydroxybenzene to prepare “varin” and such other analogs of CBG. Geraniol utilized in the examples provided herein can be replaced by other terpenols such as farnesol to prepare isoprene homologs of CBG.

### Example V Production of Cannabiqerol (CBG)

A. Olivetol (4.538 kg) is dissolved in 83 L of toluene containing p-toluenesulfonic acid monohydrate (0.359 kg) and stirred at 30° C. in a jacketed reactor. Geraniol (5.825 kg) is charged to the reactor and stirred for 1 h. The reaction mixture is quenched with saturated sodium bicarbonate (6.625 kg) and cooled to 15° C. After 30 minutes, agitation is stopped to allow phase separation. The aqueous layer is separated and discarded as waste. DI water (20 L) is charged to the reactor and mixed for 30 minutes. The aqueous layer is separated and discarded as waste. The organic solution is concentrated under vacuum to yield crude CBG concentrate (6.06 kg). The crude CBG concentrate is purified by liquid chromatography on alumina media with toluene as eluent. The purified CBG is concentrated under vacuum to yield purified CBG concentrate (2.25 kg).

B. Purified CBG concentrate (9 kg) is dissolved in n-heptane (13.8 kg) and cooled slowly to -10° C. The product slurry is filtered and washed with cold n-heptane (6.2 L). The product cake is dried under N₂ to give 3.36 kg CBG.

C. CBG crystals (3.36 kg) are dissolved in n-heptane (44.7 L) under N₂ at 40° C. The solution is cooled slowly to 28 C and held for 30 minutes. The solution is cooled slowly to 5 C and then held for 1 h. The product slurry is filtered and washed with cold heptane (6 L). The product cake is dried under N₂ to give pure CBG crystals (2.18 kg).

### Example VI Preparation and Purification of Cannabichromene

To a three-necked round bottomed flask (5 L capacity) equipped with a condenser under N2 atmosphere is added 106.3 g olivetol (0.59 mol) in 1.86 L o-xylene and the mixture is heated to 45° C. At 45° C. solution temperature, 134.71 g citral (0.88 mol) and 21.56 g t-butylamine (0.29 mol) are charged to the vessel. The mixture is heated to 130 C and hold for 2.5 hours, after which time it is cooled down to room temperature and quenched with 0.35 L of 1 M phosphoric acid. After 15 minutes, agitation is stopped to allow phase separation. The aqueous layer is separated and discarded as waste. The organic layer is then washed with 0.35 L of DI water, which is drained after 15 minutes. The organic layer is concentrate at 70° C. under full vacuum to 500 g/L of CBC.

1. A process for preparing one or more of a compound of formula (IA), (IB), and (IC): 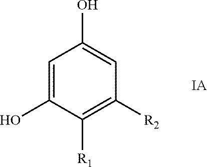 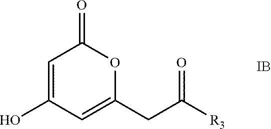 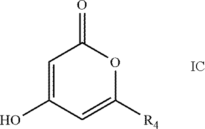
or a salt or an ester (carboxy and /or phenolic) thereof, wherein
R¹ is H or CO₂H;
each R₂, R₃, and R₄ is independently C₃-C₁₀ alkyl, C₃-C₁₀ alkenyl, or C₃-C₁₀ alkynyl, preferably, C₃-C₈ alkyl, more preferably, n-pentyl or n-propyl; the process comprising:
fermenting a recombinant microorganism comprising: a polyketide synthase and optionally a dimeric α+β barrel (DABB) protein, wherein the polyketide synthase combines an acyl-CoA and two or more malonyl-CoA to produce a polyketide and wherein the dimeric α+β barrel (DABB) protein provides the polyketide comprising a carboxylic acid,
thereby preparing one or more of a compound of formula (IA), (IB), and (IC) or the salt or the ester thereof.

2. The process of claim 1, wherein at least one compound prepared is of formula (IA).

3. The process of claim 1, wherein at least one compound prepared is of formula (IB).

4. The process of claim 1, wherein at least one compound prepared is of formula (IC).

5. The process of claim 1, wherein the acyl-CoA is Oleoyl-CoA, Palmitoleoyl-CoA, Stearoyl-CoA, Dehydrostearoyl-CoA, Oxostearoyl-CoA, Enoyl-CoA, Oxacyl-CoA, Hexanoyl-CoA, Oxohexanoyl-CoA, Butanoyl (or Butyryl)-CoA, Crotonoyl-CoA, Acetoacetyl-CoA, Pentanoyl-CoA, or Oxopentanoyl-CoA.

6. The process of claim 1, wherein the acyl-CoA is a synthetic molecule that functions similar to an acyl-CoA and is accepted by the polyketide synthase enzyme.

7. The process of claim 1, wherein the polyketide synthase is olivetol synthase (OLS) having an amino acid sequence that is at least 95%, at least 96%, at least 97%, at least 98%, or at least 99% identical with SEQ ID 1.

8. The process of claim 1, wherein the DABB protein is olivetolic acid cyclase (OAC) having an amino acid sequence that is at least 95%, at least 96%, at least 97%, at least 98%, or at least 99% identical with SEQ ID 2 or SEQ ID 4.

9. The process of claim 1, wherein the polyketide synthase shares at least 50% sequence identity with the amino acid sequence of SEQ ID 1 and whose alpha carbon backbone of its structure does not deviate by more than 1.5 Å with olivetol synthase (OLS) having the amino acid sequence of SEQ ID 1.

10. The process of claim 1, wherein the DABB protein has an amino acid sequence that is at least at least 50% identical to olivetolic acid cyclase (OAC) of SEQ ID 2 or SEQ ID 4.

11. The process of claim 1, wherein the microorganism comprises an acyl-CoA synthetase enzyme that can convert a carboxylic acid to an acyl-CoA.

12. The process of claim 1, wherein the microorganism comprises an acyl-CoA synthetase enzyme, which is CsAAE1 having an amino acid sequence of SEQ ID 3.

13. The process of claim 1, wherein the microorganism comprises an acyl-CoA synthetase enzyme having an amino acid sequence that is at least 50-75% identical with the amino acid sequence of SEQ ID 3.

14. The process of claim 1, wherein one or more phenolic hydroxy moieties of the compound of formula (IA), (IB), or (IC), or a salt thereof is esterified in vivo (or endogenously) as a result of overexpression of an arylesterase in the microorganism.

15. The process of claim 1, wherein the compound of formula (IA), (IB), or (IC) is glycosylated in vivo as a result of overexpression of a glycosylase in the microorganism.

16. The process of claim 1, wherein the microorganisms is a fungus, a bacteria, or an algae.

17. The process of claim 1, wherein the microorganism is S. cerevisiae.

18. The process of claim 1, wherein at least a part of the acyl-CoA or a salt thereof is exogenously added to a reactor where the fermenting occurs.

19. The process of claim 6, wherein the acyl-CoA like synthetic substrate or a salt thereof is exogenously added to a reactor where the fermenting occurs.

20. The process of claim 12, wherein the carboxylic acid corresponding to the acyl-CoA or a salt thereof is exogenously added to a reactor where the fermenting occurs.

21. The process of claim 1, wherein the microorganism is fermented aerobically in the presence of a water immiscible, liquid, hydrophobic phase which dissolves the one or more of a compound of formula (IA), (IB), and (IC) or the salt or ester thereof.

22. The process of claim 21, further comprising separating the hydrophobic phase from an aqueous phase comprising the microorganism, the separating comprising a first continuous centrifugation to separate the cells and a bulk of a spent broth from the hydrophobic phase, followed by a second continuous centrifugation to separate the hydrophobic phase from the remaining aqueous phase.

23. The process of claim 21, further comprising: esterifying, isoprenylating, or performing an annulation of the compound included in the hydrophobic phase, under conditions suitable to perform an esterification, isoprenylation, or annulation without the need for a solvent swap.

24. The process of claim 23, wherein the compound prepared is isoprenylated.

25. The process of claim 21, wherein the compound dissolved in the hydrophobic phase is one or both of olivetolic acid or a salt thereof and olivetol.

26. The process of claim 1, wherein hexanoic acid and optionally 3-oxooctanoic acid, 3,5-dioxodecanoic acid, or 3,5,7-trioxododecanoic acid or a salt of each thereof are exogenously supplied to the fermenter.

27. A process comprising:
aerobically fermenting a recombinant microorganism comprising: a polyketide synthase, optionally an olivetolic acid cyclase (OAC), and further optionally a hexanoyl Co-A synthetase (HCS), wherein the fermenting is performed in the presence of a water immiscible, liquid, hydrophobic phase,
to prepare one or more of: olivetolic acid or a salt or ester thereof, and olivetol or an ester thereof,
wherein the hydrophobic phase dissolves olivetolic acid or a salt or ester thereof or olivetol or an ester thereof.

28. The process of claim 27, wherein the olivetolic acid is partially or completely esterified endogenously within the microorganism to prepare the olivetolic acid ester.

29. The process of claim 27, wherein the olivetolic acid ester is prepared exogenously comprising esterifying olivetolic acid with an alcohol under conditions suitable to prepare an olivetolic acid ester.

30. The process of claim 27, wherein one or more hydroxyl or carboxylic acid moieties of olivetolic acid, olivetol, or an olivetolic acid ester are partially or completely glycosylated by the microorganism to provide glycosylated olivetolic acid, glycosylated olivetol, or glycosylated olivetolic acid ester.

31. The process of claim 27, wherein the fermentation product is acidified by addition of an acid, to maximize recovery of olivetolic acid in the hydrophobic phase.

32. The process of claim 27, wherein olivetolic acid contained in the hydrophobic phase is subjected to process conditions resulting in decarboxylation so that the olivetolic acid is converted substantially to olivetol.

33. The process of claim 27, wherein hexanoic acid and optionally 3-oxooctanoic acid, 3,5-dioxodecanoic acid or 3,5,7-trioxododecanoic acid or a salt of each thereof is exogenously supplied to a reactor where the fermenting occurs.

34. The process of claim 27, further comprising separating the hydrophobic phase from an aqueous phase. In one embodiment the separation process comprises a first continuous centrifugation to separate the cells and a bulk of a spent broth from the hydrophobic phase, followed by a second continuous centrifugation to separate the hydrophobic phase from the remaining aqueous phase.

35. The process of claim 27, further comprising isoprenylating the olivetol, olivetolic acid or a salt thereof, or the olivetolic acid ester included in the hydrophobic phase, without the need for a solvent swap, under conditions suitable to perform an isoprenylation, to prepare a cannabinoid or a mixture of cannabinoids.

36. The process of claim 27, wherein the hydrophobic phase comprises an alkane, an alcohol preferably with carbon number greater than 4 such as a C₅-C₈ alcohol, an ester, a triglyceride, a diester such as dialkyl malonate, a commercially available oil (e.g. sunflower oil, olive oil, vegetable oil or the like) or a combination thereof.

37. The process of claim 27, wherein the olivetolic acid or the salt thereof contained in the hydrophobic phase is esterified with an alcohol under conditions suitable to esterify the carboxyl moiety of olivetolic acid or a salt thereof to yield alkyl olivetolate.

38. The process of claim 37, wherein the alcohol is selected from alcohols with 2 or more carbons such as C₂-C₈ alcohols.

39. The process of claim 35, wherein the cannabinoid or one or more of the cannabinoids contained in the cannabinoid mixture include a carboxyl moiety or a salt or ester thereof, and such cannabinoids are decarboxylated under conditions suitable for decarboxylation, to prepare a decarboxylated cannabinoid.

40. The process of claim 27, wherein the olivetolic acid or the salt thereof contained in the hydrophobic phase is decarboxylated under conditions suitable for decarboxylation to provide an initial composition comprising olivetol.

41. The process of claim 40, wherein the initial composition comprising olivetol is isoprenylated under conditions suitable for isoprenylating a phenolic compound.

42. The process of claim 41, wherein the cannabinoid composition is purified, optionally hydrolyzed, and isolated to provide one or more cannabinoids.

43. The process of claim 42, wherein the cannabinoid is cannabigerolic acid (CBGA), cannabichromenic acid (CBCA), cannabinolic acid (CBNA), tetrahydrocannabinoic acid (THCA), cannabidiolic acid (CBDA), cannabigerol (CBG), cannabichromene (CBC), or cannabinol (CBN), tetrahydrocannabinol (THC), cannabidiol (CBD), or optionally a prenylogous version of the above (e.g. sesqui-CBG), or any compound that causes activation of the CB1, CB2, or TRP receptors.

44. A process comprising:
aerobically fermenting a recombinant microorganism comprising: a polyketide synthase, optionally an olivetolic acid cyclase (OAC), and further optionally butyryl Co-A synthetase, wherein the fermenting is performed in the presence of a water immiscible, liquid, hydrophobic phase;
to prepare one or more of: divarinic acid or a salt or ester thereof, and divarin,
wherein the hydrophobic phase dissolves divarinic acid or a salt or ester thereof or divarin, as they are prepared.

45. The process of claim 44, wherein the divarinic acid is partially or completely esterified endogenously within the microorganism to prepare the divarinic acid ester.

46. The process of claim 44, wherein the divarinic acid ester is prepared exogenously comprising esterifying olivetolic acid with an alcohol under conditions suitable to esterify a carboxylic acid.

47. The process of claim 44, wherein one or more hydroxyl or carboxylate moieties of divarinic acid, divarin, or divarinate esters are partially or completely glycosylated by the microorganism to provide glycosylated divarinic acid or a salt thereof, glycosylated divarin, or glycosylated divarinate ester.

48. The process of claim 44, wherein the fermentation product is acidified by addition of an acid, to maximize recovery of divarinic acid in the hydrophobic phase.

49. The process of claim 44, wherein divarinic acid contained in the hydrophobic phase is subjected to process conditions resulting in decarboxylation so that the divarinic acid is converted substantially to divarin.

50. The process of claim 44, wherein butyric acid and optionally 3-oxooctanoic acid, 3,5-dioxodecanoic acid or 3,5,7-trioxododecanoic acid or a salt of each thereof is exogenously added to a reactor where the fermenting occurs.

51. The process of claim 44, further comprising separating the hydrophobic phase from an aqueous phase, the separating comprising a first continuous centrifugation to separate the cells and the bulk of the spent broth from the hydrophobic phase, followed by a second continuous centrifugation to separate the hydrophobic phase from the remaining aqueous phase.

52. The process of claim 44, further comprising isoprenylating the divarin, divarinic acid, or the divarinic acid ester included in the hydrophobic phase, without the need for a solvent swap, under conditions suitable to perform an isoprenylation, to prepare a cannabinoid or a mixture of cannabinoids.

53. The process of claim 44, wherein the hydrophobic phase comprises an alkane, an alcohol preferably with carbon number greater than 4 such as a C₅-C₈ alcohol, an ester, a triglyceride, a diester such as dialkyl malonate, a commercially available oil (e.g. sunflower oil, olive oil, vegetable oil or the like) or a combination thereof.

54. The process of claim 44, wherein the divarinic acid or the salt thereof contained in the hydrophobic phase is esterified with an alcohol under conditions suitable for esterification to provide alkyl divarinate.

55. The process of claim 54, wherein the alcohol utilized for esterification is selected from alcohols with 2 or more carbons such as C₂-C₈ alcohols.

56. The process of claim 52, wherein the cannabinoid mixture is decarboxylated to yield a decarboxylated cannabinoid.

57. The process of claim 44, wherein the divarinic acid or the salt thereof contained in the hydrophobic phase is decarboxylated to provide an initial composition comprising divarin. Optionally, acid may be added before or during decarboxylation to protonate divarinate salts and / or catalyze the decarboxylation reaction. Optionally, the solution may be heated to increase the decarboxylation rate. Optionally, a base may be added.

58. The process of claim 57 wherein the initial composition comprising divarin is isoprenylated to provide a cannabinoid composition.

59. The process of claim 57 wherein the cannabinoid composition is purified, optionally hydrolyzed, and isolated to yield one or more cannabinoids.

60. The process of claim 59 wherein the cannabinoid is cannabigerovarinic acid (CBGVA), or cannabichromevarinic acid (CBCVA), or cannabinovarinic acid (CBNVA), or tetrahydrocannabivarinic acid (THCVA), or cannabidivarinic acid (CBDVA), or cannabigerovarin (CBGV), or cannabichromevarin (CBCV), or cannabivarin (CBNV), or tetrahydrocannabivarin (THCV), or cannabidivarin (CBDV), or any meroterpenoid compound that causes activation of the CB1, CB2, or TRP receptors.
